(19) United States
(12) Patent Application Publication
(10) Pub. No.: US 2021/0338867 A1
(43) Pub. Date: Nov. 4, 2021

(54) WEARABLE UV-C GLOVES FOR MICROBIAL DECONTAMINATION FROM SURFACES

(71) Applicant: Jeffrey S. Gibson, Van Buren, AR (US)

(72) Inventor: Jeffrey S. Gibson, Van Buren, AR (US)

(21) Appl. No.: 17/306,899

(22) Filed: May 3, 2021

Related U.S. Application Data
(60) Provisional application No. 63/018,851, filed on May 1, 2020.

(51) Int. Cl. A61L 2/10

(52) U.S. Cl. CPC A61L 2/10; A61L 2202/14; A61L 2202/16; A61L 2202/11

(57) ABSTRACT

Disclosed herein is a glove device for safely and reliably handling and decontaminating surfaces from microorganisms as well as continuously self-decontaminating subsequent to and/or during utilization thereof. Such a glove includes embedded UV-C light sources under controlled power outputs to impart decontamination/disinfection capabilities as well as protect any users thereof from potential low UV wavelength effects. Such light sources utilize a specific range of low UV radiation within the UV-C wavelengths (from 240-300 nm) generated by individual UV light emitting diodes (LEDs) with possible additional emission capabilities through fiber optics. Additionally, the LEDs extend from an external layer of water-proof, substantially nonporous, potentially IPA-resistant material, in order to allow for UV-C emissions to direct outwardly from the device for exposure to a contacted surface as well as over the entirety of the outer surface of the device itself.

### CROSS-REFERENCE TO RELATED APPLICATION

This application claims priority to copending U.S. Provisional Patent Application Ser. No. 63/018,851, filed on May 1, 2020, the entirety of which is herein incorporated by reference.

### FIELD OF THE DISCLOSURE

Disclosed herein is a glove device for safely and reliably handling and decontaminating surfaces from microorganisms (including viruses, bacteria, molds, and the like) as well as continuously self-decontaminating subsequent to and/or during utilization thereof. Such a glove includes embedded UV-C light sources under controlled power outputs to impart decontamination/disinfection capabilities as well as to protect any users thereof from potential low UV wavelength effects. Such light sources utilize a specific range of low UV radiation within the UV-C wavelengths (from 240-300 nm) generated by individual UV light emitting diodes (LEDs). The UV-C sources may be pressure-activated and/or operated by contact and/or sensed presence of a user. Such decontamination capabilities are attained through the utilization of layered glove structures with a plurality of properly spaced UV-C LEDs (light-emitting diodes) extending from an external layer of a moisture/water-resistant, non-porous, isopropyl alcohol-resistant material(s). Such a material provides a barrier to moisture droplets (thereby preventing entry thereof underneath such an outer layer) to permit thorough disinfection of the glove surface by the UV-C LEDs embedded therein and allowing for a reliably cleanable overall device to protect the user/wearer. Additionally, the embedded LEDs within the outer layer provide a suitably grippable surface effect for the user/wearer in addition to supplying disinfecting capabilities. Such an outer layer material further allows for UV-C emissions to emanate outwardly from the glove for exposure to a contacted surface as well as over the entirety of the outer surface of the glove itself. Below such an outer layer (with the UV-C LEDs spaced appropriately and provided with roughly 180 degrees of light emission therefrom for such outward and device surface exposure coverage) may be a pressure sensor in contact with a circuit (such as a flexible circuit to permit range of motion if a wearable device) and an MCU and/or timer switch or like component for programmable control of the duration and power levels undertaken by the LEDs when activated. A further inner layer may be provided, such as a fabric layer (for wicking moisture and insulating from heat generated by the LEDs when activated, thus for comfort for a glove wearer). Such a multi-layer approach with the needed nonporous (or substantially nonporous, alternatively), moisture-resistant, IPA-resistant, waterproof outer layer having the subject LEDs extending therefrom and therethrough provides the platform as noted above for such a protective and active barrier for passive cleaning and self-decontamination capability for all such target end uses that have heretofore been unexplored. Such specific gloves as well as methods of utilization thereof are also disclosed herein.

### BACKGROUND OF THE ART

The threat of contamination from microorganisms has existed for millenia. Whether through uncontrolled utilization of antibiotics, mutations, or other problematic scenarios, microbial infections have proven extremely difficult to control in certain situations. For instance, hospitals (and like environments) have suffered from potentially severe contamination of untreatable diseases, whether related to, as just some examples, Clostridia difficile (C. diff), methicillin-resistant Staphylococcus aureus (MRSA), certain strains of Escherichia coli (E. coli), and many other mutating bacteria. There exists currently a pandemic associated with coronaviruses (including COVID-19, and the like). Such examples of microbial infections have caused global concerns, leading to severe illnesses and highly unfortunate deaths within populations around the world. The best practices to currently handle such coronavirus outbreaks are quarantining in order to hopefully allow such microorganisms to lack further hosts and thus essentially die out over time. Otherwise, eradication of such microorganisms has proven extremely difficult in a widespread manner as transfer between individuals has appeared rather easy to accomplish. The above-noted bacterial strains have likewise proven hard to kill as growth and reproduction thereof is rapid and disinfection is not a simple process. Viruses, in particular, are difficult to remove due to the structures thereof, having protein strands including certain replicating RNA and DNA that are well-protected by buffy layers of lipids that bind well to surfaces as well as prevent or at least serve as obstacles to penetration of chemical/pharmacological RNA/DNA disruptors. Even more difficult to disrupt are bacteria, potentially, since such larger microorganisms may absorb more in outer layers and require more treatments for killing thereof. Similar issues exist in bacterial situations with viruses, particularly where the base organism only needs a food source to grow and reproduce, let alone such microorganisms (whether viral or bacterial, for that matter) have shown a propensity to mutate over time to evade certain chemical and/or pharmacological treatments. As such, many microorganisms have attained levels of resistance to certain pharmacological treatments, leading to microbes that replicate quickly and are not easily destroyed (such as, again, at the RNA/DNA level). Additionally, as alluded to above, the ability for such microorganisms to mutate in order to become immune to certain treatments (particularly chemical in nature) leaves limited options as to the control and/or eradication of such microbial concerns.

As such, the most reliable manner of treating such microorganisms may be the utilization of light, particularly outside the visible spectrum within the ultraviolet regions. UV light has been shown to disrupt any number of cellular structures, whether at the cellular or tissue level. Certain portions of the UV spectrum, UV-A and UV-B in particular, are well known for causing mammalian skin, for example, to gradually alter color and, at times shape, going so far as to mutating at certain phases to cause cancers (carcinomas and melanomas, at least). Far UV light (100-200 nm wavelengths) has been considered for such microbe disruptions, however such low wavelength light seems to actually provide too fast a capability, actually appearing to allow the disrupted DNA/RNA bonds to repair and/or reconnect after cleaving, thus allowing for the proteins to remain effective with only a slight possibility of disruption. Increased power levels and longer exposure may permit far UV some better results in this manner, except that such issues require power levels that can cause far worse results as human exposure times and power levels typically result in greater harm than benefit.

To the contrary, UV-C light is within a much lower range of wavelengths on the UV spectrum (from roughly 180-300 nm) and, in similar fashion, is well known to cause cellular disruptions upon exposure, even at exposure times of very rapid duration (seconds and lower, for instance). A second or so of exposure, for instance, is known to cause burning to human skin, particularly at an elevated (and typically utilized) power level (100 watts, even as low as 100 mW), thus militating against widespread use. As a result, there is a need to provide certain controls and limits for UV-C light generators and lamps/lights to ensure such undesirable skin problems are avoided. Similarly, as with any UV light source, it is important to avoid eye exposure directly to such wavelengths as they have been known to cause intense burning and potential ophthalmic retinal damage (sometimes inoperable and permanent) if too prolonged an exposure occurs. Corneal absorption of UV rays may occur, but if the intensity and power levels of UV-C emissions are excessive, such a natural defense will not be of actual help. Even with such potential issues, the ability of UV-C light, in particular, to create disruptions of microorganism RNA/DNA is important as an alternative to standard chemical/pharmacological treatments. Coronaviruses, and COVID-19 as one definitive example, have rather transparent and thin buffy lipid layers that may be penetrated easily and well and thus allow for such low wavelength UV light to access to basically destroy the proteins therein, preventing replication and thus effectively killing the virus. This capability may be effective with as little as 0.2 mW of power from a distance of about 3.0 cm, in fact, allowing for a potential remedy to such a quickly replicating microorganism.

The basic problems with past UV-C applications have been the lack of protection for users in a manner that allows for controlled light emissions for microorganism exposure (and thus disruption of proteins, etc.) but with limited to no exposure of such potentially harmful UV-C light to the human user her- or him-self. Additionally, with the power levels needed to generate such UV-C light emissions at a distance from target surfaces, the generation of significant heat therefrom is harmful as well to a user, particularly if the light source is manipulated by hand for such a disinfecting purpose. For example, wand devices, and, for that matter, uncovered UV lamps, have been utilized in the past to provide some degree of UV treatment of microbes within certain environments (particularly within a limited atmosphere). Such devices, unfortunately, are provided at much too high a power level for UV-C to be safe for environmental exposure purposes. In other words, the power levels typically associated with lamps and wands necessarily are of significantly high-power levels in order to provide distance exposure kill capabilities for environmental treatments (100 watts, or as low as possibly 10 watts); for UV-C emissions, such power levels, though effective for microbial kill in such situations, is far too great for human skin and eye exposures to be of any interest for continuous usage. As such, these UV-C lamps/wands do not generally include any further protections for users from exposure thereto. Additionally, such wands/lamps require significant distances for decontamination purposes, except for the chance that a user scans such a UV light source over a surface. In such situations, however, distances and, for that matter, haphazard applications through random movements by the user, do not allow for treatment uniformity, leaving the target surface susceptible to further contamination thereafter due to a lack of complete and overall UV light coverage. A significantly close and uniform exposure distance (within a few centimeters, for instance) rather than a stationary light source or waved/moved UV wand (again lacking exposure protections for a user) would provide an overall benefit as needed for reliable and safe microbial eradication. To date, however, such a capability has not been provided within the pertinent art.

There thus is needed a more robust manner of providing surface decontaminations, specifically as it concerns viral and bacterial, at least, microorganisms that may reside thereupon and may be easily transferred to human hosts therefrom. Such a method of surface disinfecting/decontaminating may include a device that may be manipulated easily by a user, may be contacted with, wiped across, and/or otherwise directed toward, at close proximity, such a target surface, and provides protections from UV-C exposure to a user's or bystander's eyes and skin. To such a degree, then, the power potentially required to effectuate such microbe decontamination/disinfection is related to the distance required for microbe killing (RNA/DNA disruption, for instance), referred to as the radiant flux of the UV-C light source, and may be properly monitored to ensure maximum killing effect on microbes with a reduced propensity of, for instance, excess heat exposure for a user, particularly if such a device is hand-held and placed in such close proximity to the target surface. To date, unfortunately, there has been nothing provided within the art of interest (target surface decontamination, for example) that utilizes any type of device that meets such stringent requirements. Of interest may be a device that accords not only self-cleaning during actual use, but also passive cleaning capability of a target surface when utilized in relation to any type of potentially infected substrate (such as a glove having embedded UV-C light sources that allows for range of motion, gripping/carrying/wiping of surfaces, and thus functions to not only protect the user from infection, but transfers, passively, such decontamination capabilities to substrates/surfaces contacted therewith during use). Additionally, then, such a surface decontamination method may also include more active cleaning operations utilizing self-moving devices with UV-C light sources incorporated therein for directed, close proximity applications without need for either user manual controls and/or direct visibility of any UV-C light emissions for such a method to commence. To date, however, such a potentially desirable methodology has yet to be undertaken in such a fashion, particularly within the UV-C spectrum, ostensibly due to the aforementioned difficulties with human interaction with such low UV light treatments and the lack of controlled UV-C device activities that would be needed to overcome such human exposure issues.

Furthermore, any such device for UV-C emissions-based decontamination may be problematic with a material that allows moisture past the outer layer (at least in an appreciable amount and/or manner) since water/moisture may cause shorts within the electrical components thereof and since microorganisms could congregate within water droplets and reside in a position unexposed to such surface UV-C LEDs. Thus, a sufficient water barrier (nonporous, or substantially nonporous, to at least prevent water droplet penetration, with water-proof/moisture-resistance qualities as well) is needed to avoid such a deleterious result. Also, isopropyl alcohol is utilized for various reasons in a sterile (or preferably sterile) environment, particularly with patients with wounds that require disinfection with such a liquid (of course, the widespread utilization of hand sanitizers with such gloves or in the proximity thereof could also affect such outer layers, as well, thus necessitating IPA-resistance properties). The utilization of gloves in such a setting is quite typical and thus such a material constituting the disclosed gloves herein must also exhibit sufficient IPA resistance to remain dimensionally stable and thus effective for repeated and continuous utilization. As well, a material that does not prevent moisture from contacting circuitry and LED sources may prove damaging to the device.

A properly small and thin device, at least in terms of layers of materials, to accord flexibility for a user without appreciable level of tearing, breaking or otherwise compromising the dimensional stability thereof, would likewise be attractive for such an important purpose. To date, the industries involved are devoid of such a possible system for microbe decontamination.

The present disclosure, however, overcomes such prior deficiencies and provides a suitable, reliable, and safe platform of different types of glove devices and methods of utilization thereof for target surface decontamination/disinfecting purposes as well as continuous self-decontamination capabilities.

### SUMMARY OF THE DISCLOSURE

To overcome the above-noted deficiencies exhibited by standard high power level UV-C wands and lamps, it has been realized that devices of different types and structures, as well as for different target surfaces, may provide the necessary level of microbial kill while protecting humans from skin and eye exposure possibilities. To that end, embodiments provided herein are directed to a platform of UV-C LED light sources which may be programmable, thus enhancing the UV-C power requirement to provide microorganism kill rates at lower power levels. Such light sources may thus operate within ranges of power and generally within a wavelength range from 240-300, preferably from 240-280 nm, more preferably from 250-280 nm, potentially most preferably about 254, within the UV-C spectrum, at least. Such wavelengths have now been found to accord the highest level of viral and bacterial disruption while allowing for power levels to be set at proper measures to alleviate any potential harm to a human user (if, for instance, such a device is hand-held or operated to any degree requiring human skin to be within a certain distance therefrom the light source itself) as well as in a suitable configuration to reduce any propensity for eye exposure by such a human user and/or bystander during utilization.

These UV-C LED sources are embedded within a multi-layer glove structure that covers the entirety of a user's/wearer's hand (with a pair covering both hands entirely) in order to provide both glove surface self-decontamination capability as well as external surface contact disinfecting potential (with sufficient contact time when such LEDs are properly lit). The glove devices disclosed herein will thus include an outer layer for LED extension therefrom at the glove surface that exhibits, as noted above, certain physical properties. These properties include, without limitation, a moisture-resistant/water-proof barrier material (that prevents flow of water and/or moisture to any degree from passing through such a surface to inner layers thereof), that is nonporous (or substantially nonporous, at least to the point that water droplets cannot penetrate the surface thereof) and is further IPA-resistant to prevent disintegration of such moisture barrier gloves as well as possible electronics therein upon exposure thereto.

Such disclosed and novel gloves thus include an outer layer material that provides an effective cover into which embedded UV-C emission sources (LEDs, as examples), as well as a power source and MCU or like component to program/control UV-C emission times, durations, and power levels. Such glove devices would also thus include a type of component that allows for determination of pressure in order to activate either the entirety of the UV-C source therefor or selected discrete areas thereof within the device. In this manner, then, the ability to provide decontamination upon pressure indication allows for the UV-C source to activate and provide disinfection upon contact or close proximity location and, if desired, for a certain duration of UV-C emission thereover. This permits the device cleaning capability of a contacted surface, certainly, as well as continued sequential cleaning of the glove surface thereafter such contact is made (to, as noted herein, create a continuous disinfection device for both contacted surfaces and itself). In this manner, such a glove exhibits a capability of decontamination itself for a duration after such activation to best ensure such a glove is free from contamination sufficiently to prevent any infection therefrom.

This disclosure thus may encompass, at least, a wearable glove device comprising a plurality of light emitting diodes embedded therein to provide external and surface exposure to UV-C radiation between 240 and 300 nm wavelengths. Such a disclosed glove further comprises an external surface material through which said plurality of light emitting diodes extend outwardly, said material being waterproof, cut-resistant, and exhibiting a tensile strength that may withstand shear pressure applications, tear pressure applications, and the like, associated with standard usages thereof (thus, at least about 5,000 psi and as high as about 30,000 psi). Such an outer layer of said disclosed glove thus also provides, as noted above, a moisture-resistant, water-proof, IPA-resistant, physical result (with substantially nonporous materials). Such a glove device may further comprise at least one control component selected from the group of at least one flexible circuit, at least one MCU, and a combination thereof, wherein said at least one control component is programmable for control of duration of UV-C emissions, control of UV-C light source power levels, and control of activation of UV-C light sources in relation to pressure application on a surface by a user. Additionally, for benefit of a wearer, such a glove device may also comprise an inner layer (at and in contact with the user's skin, in other words) of moisture wicking and/or heat shielding material for comfort and/or protection of the wearer (such as fabric, including, without limitation, cotton, poly/cotton blends, and the like). Furthermore, such a glove device may comprise a pressure sensor component underneath said external material and above said inner layer material (such as a pressure layer connected with a single flexible circuit and/or MCU, or individual sensors associated with each LED source and individual circuits for activation purposes). The LEDs provide a base UV-C light source (of course, any other such UV-C light source may be employed, but LEDs are particularly suited for such a glove device due to size and facility of implementation, and thus are potentially preferred).

To accomplish such results, the disclosed glove device herein includes the utilization of a UV-C emission source (between 240 and 300 nm, preferably from about 240-280 nm, more preferably from about 250-280 nm, and most preferably from about 254-280 nm (with 254, 260, and 280 nm possibly further preferred). Additionally, these UV-C sources are provided as LED structures in order to allow for controlled trajectories of the emissions thereof as well as to control the power levels needed for robust and effective microbial decontamination results as well as complete coverage of the device in terms of UV-C emissions for glove device self-decontamination purposes. A power source is thus also of necessity, such as a battery pack, capacitor, and the like, that may be rechargeable as needed, and provides the necessary wattage for such UV-C emissions for microbial exposure (and thus kill rates). Furthermore, the disclosed device must include a means for control of UV-C source power levels, activation and deactivation operations, and time duration of active emissions upon activation (and until deactivation). For this purpose, an MCU or circuit board (such as, for maneuverability, if necessary, a flexible circuit board that will allow for electrical contact and control while permitting free range of movement for the user/wearer, again, as needed for such a possible end use) as noted above, may be present within the device and programmed to act and react appropriately in relation to activation and deactivation operations as well as power levels exhibited by such UV-C sources and for durations that accord sufficient kill rate capabilities. Also required for such device operations and complete decontamination capabilities, particularly as it concerns effective UV-C emission exposure to contacted surfaces as well as device surfaces in total, is a surface material that allows for sufficient levels of UV-C emissions, is waterproof (to protect electronic components from potentially damaging moisture as well as to prevent infiltration and/or penetration of microorganisms within external water droplets), exhibits a high tensile strength to prevent deleterious rips, tears, and/or breakages thereof that may compromise the effectiveness of the system as a whole, and may further exhibit IPA-resistance for dimensional stability when utilized in the proximity, at least, of such a disinfecting solvent. Such a material is needed to impart the needed UV-C exposure capabilities of the glove through a substantially uniform and nonporous (and, in certain embodiments, possibly a smooth and/or reflective) surface that surrounds portions of such UV-C sources (LEDs) but safely and sufficiently seals such UV-C components to prevent undesirable moisture from introduction thereunder from the glove surface. The outer layer material thereof thus allows for UV-C emissions to emanate from the glove device as well as shine/emit over the glove surface to ensure exposure to any microbes on the device surface and/or contacted surface to which the device may be applied as the UV-C sources are activated. As noted above, any large sized pores at or on the glove surface may cause problems with collected water droplets with microorganisms therein that may infiltrate within discrete areas beyond the reach of the UV-C LEDs embedded within the glove surface, thus limiting the capability for full device surface decontamination (and such moisture could harm the electrical components therein, too). A lack of moisture-resistance and/or water-proofing would likewise create problems as excessive moisture/water may deliver microorganisms to the glove surface that may then congregate and multiply if not reached by the LED emissions. In this manner, then, such glove devices are functional for proper decontamination capabilities with low power levels which favorably and significantly reduces the chances of exposure to a user/wearer or other person in close proximity thereto by allowing for lower power levels for sufficient UV-C LED killing potential (sufficient power and sufficient exposure, as an example). Any power level increase could compromise the safety aspects of the overall glove device/system disclosed herein. Additionally, then, the outer layer material must retain its dimensional structure and stability while in use to best ensure, again, that no moisture/water, etc., or IPA, for that matter, can infiltrate below the outer layer itself. Thus, a high tensile strength (at least 5,000 psi, as one low level example, again, as noted above, lower tensile strength materials may be present if such may withstand dimensional loss, such as tearing, shearing, etc., during typical end use operation, preferably at least 10,000 psi, more preferably at least 20,000 psi, and potentially more preferably about 28,000 psi) polymeric material is needed for such a purpose. As examples, potentially preferred, include polytetrafluoroethane polymers (such as GORE-TEX), flashspun highly-oriented polyethylene fibers (such as TYVEK), biaxially oriented polyethylene terephthalate (such as MYLAR, MELINEX, and HOSTAPHAN), styrene-butadiene block copolymer structures (such as KRATON), and other like materials. Such are all moisture-resistant, IPA-resistant, and substantially nonporous (prevents water droplets from penetrating); some may be further treated, such as through metallization, including, without limitation, aluminized coatings and other metallic coating/integration (including, without limitation, gold, silver, platinum, and the like, transition metals). Furthermore, for reflectivity as a further possible property thereof, the outer layer material may include a metallized coating or a likewise structural presence. Such a metallized component imparts reflectivity for the material as a metallic presence accords a non-absorptive quality thereto. The reflective material may be provided in rolls and provided with openings (punched, needled, etc.) and either placed over suitable UV-C sources or such UV-C sources introduced therethrough; in either option, the UV-C sources (LEDs) extend from the reflective material for surface emission capability. If desired, as well, such a material may be supplied around individual UV-C source extensions, rather than completely populating a single layer alone. As long as a sufficiently nonporous material (to prevent moisture penetration, at least) is present imparting a surface that will allow for the UV-C LEDs to decontaminate the glove surface. A reflective material may aid in emitting such UV-C LED lights across a region of the subject device surface (and sufficient amounts of such UV-C sources are present for surface decontamination entirely if all such sources are activated, such a reflective material may be present in any such way to ensure target surface disinfection is possible). Additionally, however, such a material in this manner also imparts electrical conductivity that allows for facilitation of circuits between a power source, an MCU, and a pressure sensor for the entire device to function properly and easily. Additionally, the system and thus the subject glove device may also include layers beneath the external UV-C source/outer layer material surface portion and the pressure sensor portion, including, without limitation, a lower waterproof material (such as a rubber, rubber-like, or like insulator material, the same reflective material as noted above, and any other like waterproof material, including possible waterproofed fabrics, as non-limiting examples), and a lower layered material that, depending on the end use, may impart wicking, heat-shielding, cushioning, or other like properties to the device. Such a lower material may thus include a wicking fabric (thin cotton for an internal glove component, for instance, that provides a manner of removing sweat from the wearer's hand, provides general comfort to the wearer, and acts as a potential heat shield to reduce potential harmful or uncomfortable effects of heat generated by the UV-C source during activation thereof), a cushioning foam, foam rubber, and the like (to act as a heat shield/insulator as well as to reduce pressure on the external LEDs as the device may be pressed on a contacted surface). Certainly, it should be well understood that a user may don an initial hand cover, such as, without limitation, a latex or rubber glove, prior to placement of the glove device disclosed herein, if desired.

Such a multi-layered device thus can be provided in any number of structures all with the capability of according such desired and effective UV-C emission exposure to a contacted surface and/or its own surface for a reliable, safe, and effective manner of decontamination of any number of surfaces and continuous disinfection of itself.

As it concerns the MCU capabilities described above, such activation/deactivation is provided through the utilization of different types of sensor components, as outlined above. Basically, a pressure sensor, which may be provided in relation to each UV-C LED location (which may include an LED as its base UV-C source), may be utilized to activate the UV-C source upon depression or other action in relation thereto. Thus, as one non-limiting example, a user may have a glove with multiple LEDs present and a pressure sensor component within an internal layer of such a glove. Upon any deformation of such a sensor layer, the MCU may then activate the LEDs thereon at the glove surface, indicating the glove is being utilized to contact a certain surface (such as, lift a box, touch a table or chair, grab a steering wheel, as non-limiting examples). Such a sensor may then return to its normal state thereafter in order to indicate the external contact has ended and thus the MCU may then deactivate the UV-C source until the pressure sensor is deformed at a later time (and then the MCU may then activate the LEDs again, and so on). Alternatively, the MCU may sense such a pressure deformation signal and activate the LEDs for a set duration of time (from 2 seconds up to, for example, 4 minutes), at which time deactivation is programmed and occurs. Such pressure sensing/deformation may also be programmed to allow for such LEDs to remain lit after pressure is not sensed after initial deformation occurs, as well. Further pressure sensing in relation to already lit LEDs may then extend the duration of LED activation for the full programmed timeframe in relation to such a second (or subsequent) sensor deformation. If desired, however, the system may allow for localized pressure sensor deformation and the MCU may only activate a certain LED or set (or collection) of LEDs, such as within a certain localized geography of the device (within the range of actual contact of the device or within a range local area thereof) at which point the MCU may activate such a limited amount of LEDs for decontamination either as long as contact is made or, as above, for a certain duration as programmed, etc. In this manner, then, the device may include a single MCU for all such control/programming purposes, or the device may include a plurality of MCUs in relation to certain numbers of the LEDs present for such localized controls. Additionally, IR sensors may also be included to sense human skin presence in order to, if needed, control power levels of the UV-C sources and/or to deactivate such a device if skin is too close (in order, either way, to best ensure, if needed, that damage to human skin or eyes, for that matter, is reduced significantly through such capabilities). If desired, the system disclosed herein, and thus the glove device as disclosed in myriad possible ways, may also include at least one accelerometer to allow for positional sensor capabilities as a manner of indicating the user's or device's orientation as activated in relation to a contacted surface or to itself. Furthermore, another possible inclusion is a Bluetooth and/or RFID component that allows for the system and/or device to communicate with any number of external programs/apps/others in relation to any number of monitored considerations (telemetry, location monitoring, potential microbial presence level increases/decreases, power levels utilized, basically any metric desired for measurability and/or safety monitoring and/or any other capability of interest). Such tracking and communication capability also allow for network tracking and other like issues, as well, for monitoring of usage of multiple devices to potentially assess hot spots of microbial activity that may require further involvement.

With the glove devices as extensively described above, again, such may activate/deactivate in different ways and manners, but the ability to impart such decontamination/disinfection capabilities are rooted within the utilization of UV-C sources coupled with water-proof/moisture-resistant, substantially nonporous, IPA-resistant, potentially, though not necessarily, reflective, high tensile strength surface materials and sensors and MCUs with suitable power sources, for such automated cleaning results. Such a surface (outer layer) material may also preferably be smooth (substantially smooth, at least) to avoid any wrinkles, bumps, crevices, or contours that may create surfaces formations within which moisture may collect and the LEDs cannot sufficiently emit light for sanitation/decontamination thereof. The utilization of smooth, reflective materials with the UV-C sources in this manner may further allow for sufficient emissions to cause microbial DNA/RNA disruptions as needed for such decontamination purposes while safely applying such close proximity UV-C light with control to limit the power levels required for maximum kill rates, thereby imparting a safe and effective process allowing human utilization without undue or appreciable harm to skin or eyes as a result. Such LEDs may be of any suitable type that allow for UV-C emissions (such as with silver and silica bulbs, particularly with emanations at a full 180 degrees from the source). Since the distance of transmission from the light source (UV-C LED, for instance) is relatively short, the power required for such UV-C emissions transfer therethrough is relatively low and does not require a significant increase over that needed for safe decontamination levels. The further presence of a possible reflective surface material (MYLAR, for example) allows for increased emissions outwardly for surface and exposed object surface decontamination purposes.

Thus, in each of these possible alternatives within the overarching surface cleaning platform, the device accords sufficient UV-C cleaning/killing power with, again, proper safeguards in place to protect a user and/or bystander from any unwanted exposure to such low wavelength light sources.

### BRIEF DESCRIPTION OF THE DRAWINGS

FIG. 1 shows a graphical representation and explanation of the efficacy of utilizing UV-C sources for viral kill capabilities.

FIG. 2 shows a possible embodiment through a cross-sectional representation of a multi-layer device with UV-C LED sources for a glove or like clothing article.

FIG. 3 shows a possible embodiment through a cross-sectional representation of a multi-layer device with UV-C optical fiber sources for a decontamination article.

FIG. 4 shows a possible embodiment through a cross-sectional representation of a device.

FIG. 5 shows a possible embodiment through a different cross-sectional representation of a device.

FIG. 6 shows a possible embodiment through a different cross-sectional representation of a device.

FIG. 7 shows a possible embodiment of a glove device.

FIGS. 8 and 9 show a possible embodiment of a different glove device.

FIGS. 10 and 11 show a possible embodiment of a different glove device.

FIG. 12 shows a flow chart for an embodiment of a potential glove device system.

### DESCRIPTION OF THE DRAWINGS AND PREFERRED EMBODIMENTS

As noted above, the overarching platform for UV-C microorganism treatment capabilities covers a range of different devices/articles. Without any limitation intended, the following descriptions present a number of different systems/devices that accord such antimicrobial capabilities while ensuring safety for users simultaneously.

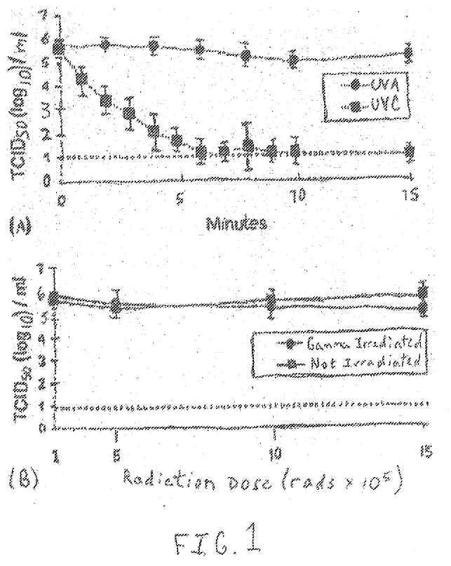

FIG. 1 of the drawings provides a graphical representation of the capability of a particular comparison of coronavirus eradication between UV-C, UV-A, gamma irradiation, and no irradiation. The platform disclosed herein includes the utilization of UV-C LED sources that generate a power that shows effective coronavirus penetration and thus disruption of RNA/DNA within the protein possible embodiment of a glove device thereof to prevent replication (effectively causing such a microorganism to remain solitary and therefore die off as the bonds within the RNA and/or DNA thereof are broken). This FIG. 1 shows a distance of 3 cm from a coronavirus treated surface with a power level of 4016 μW/cm² as an example of efficacy in killing a coronavirus. The comparative UV-A and gamma irradiation attempts appeared to leave the subject coronavirus intact at similar power levels. Most interesting of all was that the lack of any irradiation left similar results as for the UV-A and gamma irradiation samples. These FIG. 1 results thus show the capabilities of utilizing a certain power of UV-C (254 nm) wavelength light sources within 3 cm of a coronavirus sample surface. Such efficacy is extremely important, and thus the knowledge that power levels and distances considerations (radiant flux measurements) allows for proper treatment regimens to be developed. Additionally, the time required to evince effectiveness as to coronavirus kill is relatively of short duration, particularly at 3 cm distance. Closer distances and higher power levels allows for quicker eradication in combination; alternatively, even with closer distance alone, marked improvements are possible as well. This disclosure thus provides different manners of utilizing such knowledge for coronavirus (and other microorganism) eradication methods and devices that accord users such effective capabilities while simultaneously providing sufficient protection for hand manipulation and control thereof. Such a possibility in the coronavirus treatment industry, at least, has yet to be provided in such a manner, opening up significant possibilities of improving safety and protections from such potentially deadly microorganisms through simple cleaning methods and processes. Considering the potential for depletion of sanitizing formulations and fluids, let alone the possibility of viral and/or bacterial mutations to grow immunity to such treatments, the safe and reliable utilization of UV-C for such eradication efforts is of substantial benefit.

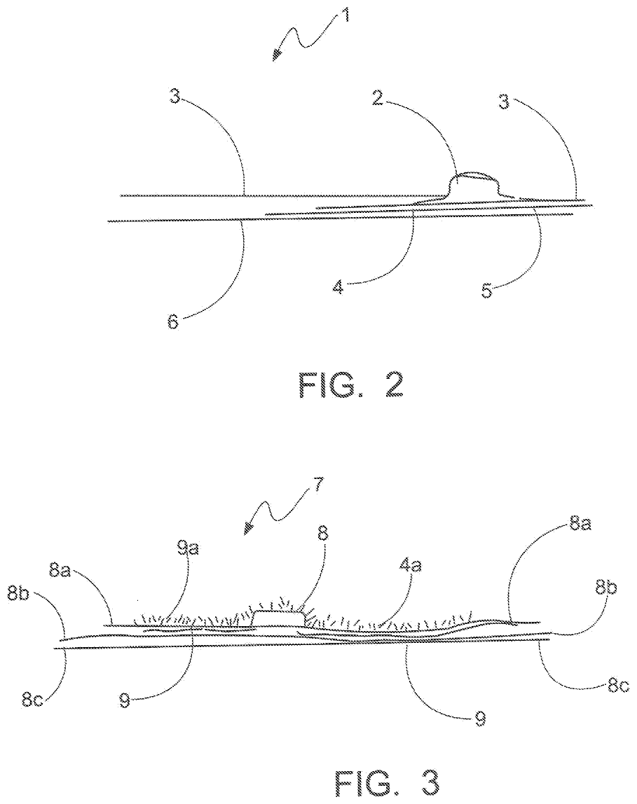

FIG. 2 shows a multi-layer structure of a possible embodiment of a device disclosed herein 1 including UV-C LEDs 2 extending outward from a Mylar surface 3. Such a configuration allows for the Mylar to reflect the emission from the LED outwardly for other surface exposure/contact as well as across the surface of the Mylar itself for disinfection thereof. Below are a pressure pad 4 for sensor communication as to deformation and activation capabilities, a lower Mylar layer 5 for moisture barrier purposes from a cotton bottom layer 6 present within a glove for comfort, heat-shielding, and moisture wicking.

FIG. 3 shows a different embodiment multi-layer structure 7 with a single UV-C LED 8 (although more than one may be present, even a pod of 2-4, for instance, within a region of a device, if desired) that are covered with a Mylar material 8A. A pressure pad 8B is present for sensor purposes as above, as is a lower layer for moisture barrier and/or conductivity as needed.

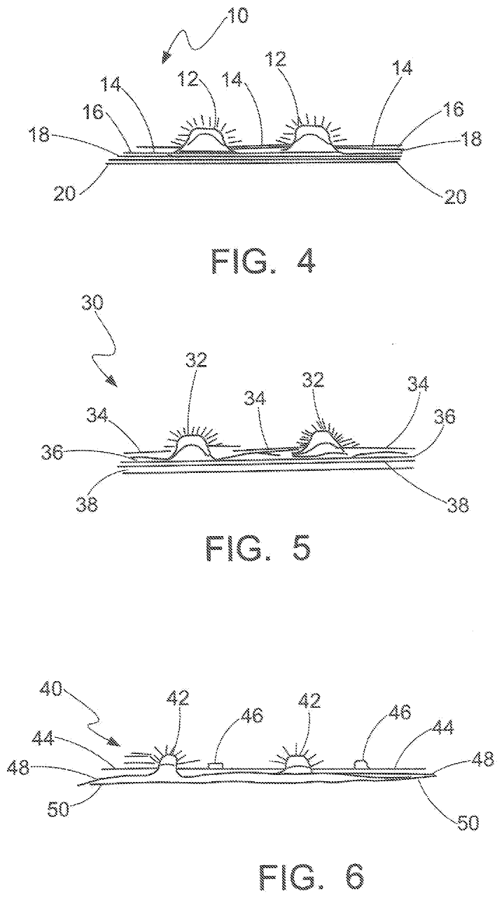

FIG. 4 shows an embodiment structure 10 with UV-C LEDs 12, a Mylar layer 14 (through which the LEDs 12 extend), a pressure sensor 16, a second lower moisture barrier layer 18 (could be Mylar, rubber, etc.), and a lower layer 20 for comfort (polyester, rubber, etc.). FIG. 5 shows a different embodiment structure 30 with UV-C LEDs 32 extending through a Mylar layer 34, a pressure sensor layer 36, and a cushion layer 38 (such as a foam rubber). FIG. 6 shows another possible embodiment structure 40 including UV-C LEDs 42 extending from a Mylar layer 44, photoelectric cells 46, a lower barrier layer 48, and a lower roughened layer for surface retention purposes. All of these structures 10, 30, 40 show device surface capabilities for decontamination of device surfaces layers 14, 34, 44 by the UV-C sources when activated.

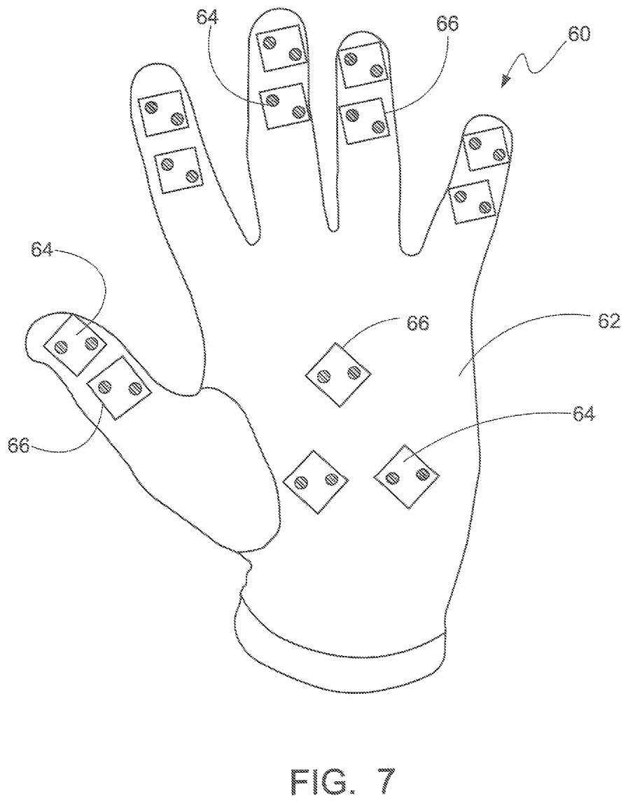

FIG. 7 shows a glove 60 with strategic layout having an outer layer 62 with embedded LEDs 64, 66 provided in pairs on the outer layer 62. The LEDs are provided with wavelengths at either 260 nm at 10 mW/cm² or 280 nm at 12 mW/cm² for maximum kill and protective power rates. (The kill wavelengths in this respect may be based on different microorganisms for kill rates; in this situation they are based on a virus equivalent). 280 nm light spectrum showed the best efficacy of log₁₀ inactivation but significantly less inactivation efficacy than that of 260 nm irradiation (i.e., 1.1 vs. 1.6 log₁₀ reduction for 5 mJ/cm² of UV fluence, P=0.01). At 280 nm light spectrum, the other viruses showed relatively low performance with log₁₀ reduction range of 0.5-0.8. The 5 mJ/cm² of UV dose using 260 nm LED can provide at least 1-log₁₀ inactivation of all the enteroviruses. Preferred dose in 5 minutes is 25 mJ/cm². For 280 nm dose you need 4 times the dose. Measured output at 280 nm is 12.5 mJ/cm². Minimum would be 4 diodes per cm2. Optimal would be 4 diodes per cm² with an output of 10 mW per diode at 280 nm. At the same dose one would need 2 diodes at 10 mW/cm² over 5 minutes with a log deactivation rate utilizing 260 nm LEDs. It may require 4 diodes at 10 mW/cm² per LED over five minutes with a log deactivation over 5 minutes utilizing 280 nm LEDs or, alternatively, 2 diodes diagonal offset at 12 mW per LED. Since the deactivation of virus is logarithmic one may still have significant deactivation within a minute or some, as well.

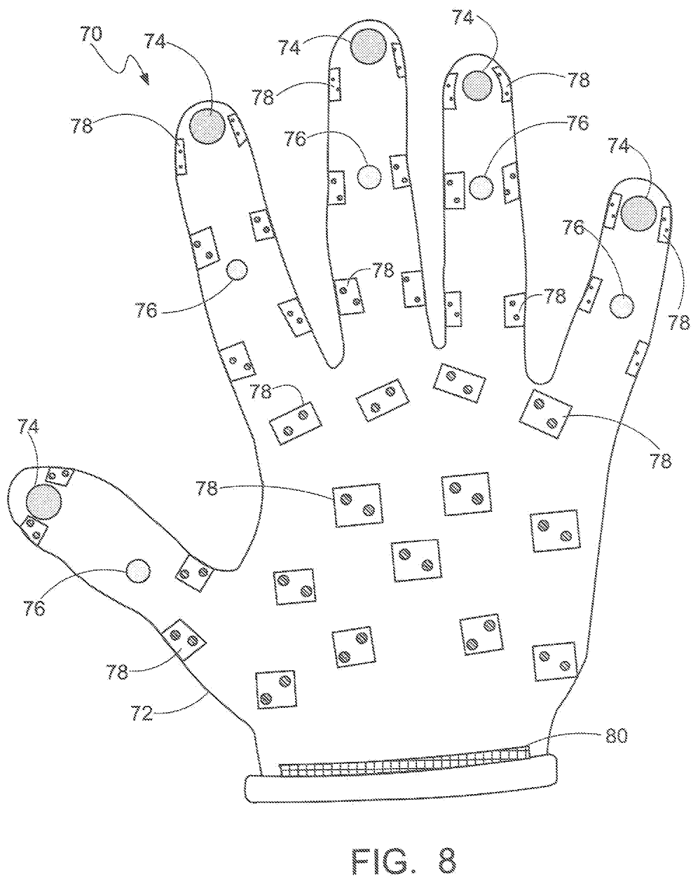

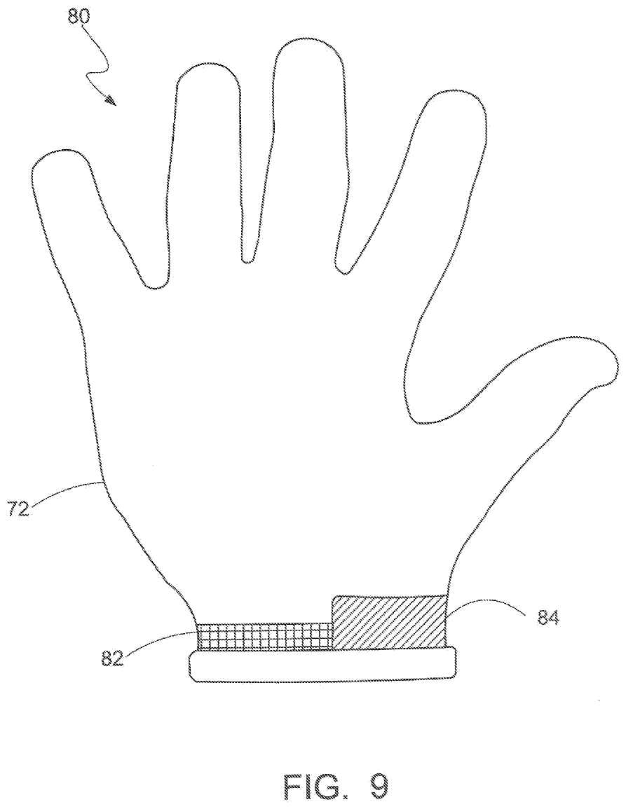

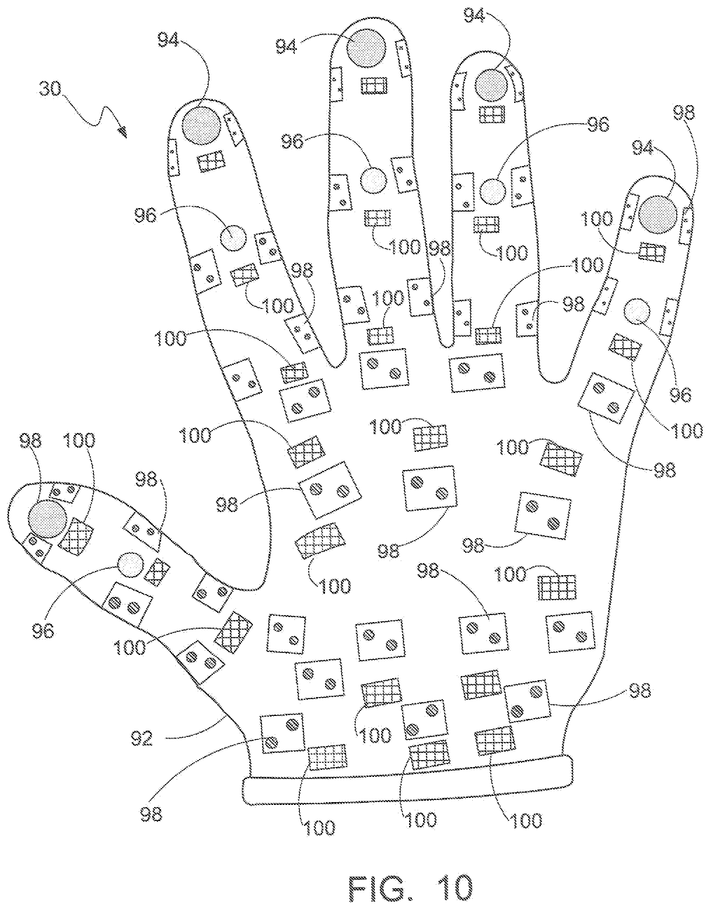

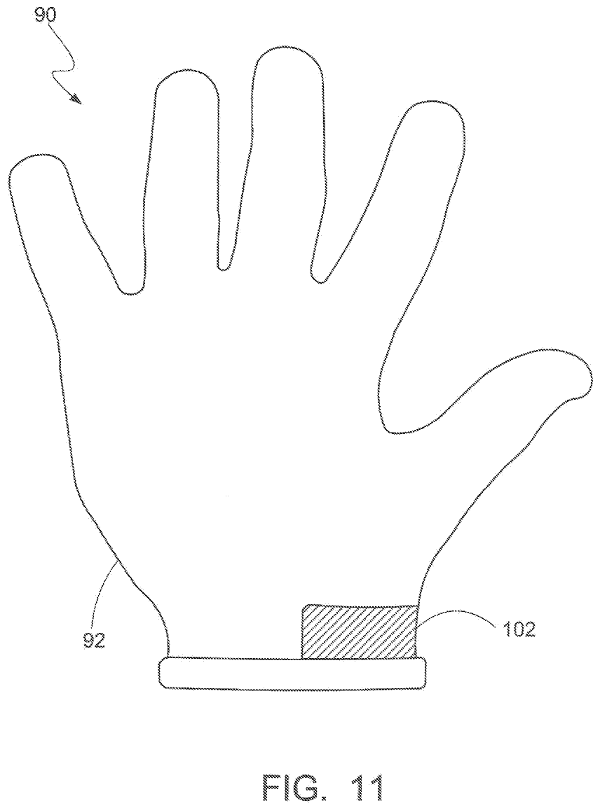

FIGS. 8, 9, 10, and 11 show glove embodiments 70, 90 in relation to the disclosure herein. In FIGS. 8 and 9, the glove 70 includes an MCU 80 near the wrist with multiple UV-C LEDs 78 to cover the entirety of the palmar regions thereof (where contact with surfaces and objects typically occur). Included are cut-outs 74, 76 for tactile sensation capabilities and circuit locations. A power source 84 is also present with a further circuit board 82 for communication between components. Sensors underneath (as in FIG. 2, at least, above) allow for contact with a surface to activate the MCU to operate the LEDs 78 for decontamination of a target surface/object. In FIGS. 10 and 11, a similar approach is followed with the glove 90 including multiple UV-C LEDs 98 and cutouts 94, 96 for tactile purposes, as well as multiple circuit boards 100 for localized controls (and thus activation at specific LEDs 98 as sensors are deformed through contact) A power source 102 allows for such activation as the circuits indicate. If desired, either glove structure 70, 90 may also include LEDs or fiber optics (or both) on the distal sides thereof to allow for complete decontamination of the gloves continuously. Additionally, such a fiber optics outlay may be implemented instead of solely LEDs, if desired.

Furthermore, then, the glove may include an inner layer of a fabric for comfort to the wearer/user, whether within the fingers or within the palmar region of the user's hand. The distal part of the glove may be outfitted with a further flexible circuit board as well as a power source (rechargeable battery, for example, as non-limiting). As the power levels for such LED lights and IR sensors, for that matter, is extremely low, recharging may not require a significant amount of time. Such rechargeability may be undertaken with an electrical cord plug-in device, USB port structure, or even placement of the glove on a recharging station. The circuit board may also include a monitoring capability to track the power levels and possible replacement needs of UV-C light sources on occasion.

Such a UV-C light emitting glove may be utilized by a user/wearer to wipe/clean surfaces or grip/carry articles as needed with any contact with other surfaces or articles imparting microorganism disinfection/decontamination through passive activity (any contact imparts such results, in other words) with active capabilities through actual movement of the glove over any target surface. With the gloves further providing self-decontamination as the LEDs extend through an outer layer or simply from the gloves themselves and thus cover the entirety of the outer surface thereof as well as any targeted surface/article simultaneously, these gloves may be provided as a complete means to ensure decontamination continuously for effective microorganism kill purposes. The extended LEDs also provide grip properties for a user due to extended structures thereof and their close proximity to one another as embedded therein. Such may make it easier to grip external surfaces for carrying, etc.

Such glove devices may be sized for any type of user (hand size, finger length, etc.) as well as provided with a lower end leading as far as the user's elbow (with UV-C light sources present within the entirety thereof to such a distance, if desired). Such an overall microbial (virus, bacterial, etc.) decontamination glove device thus allows for removal of a significant transmission vector for such potentially harmful, if not deadly, infectious organisms, namely a person's hand or hands. Additionally, the ability to control the power levels associated with the UV-C light source(s) involved, such a device may be attenuated to target certain types of microbes, rather than all. In such a manner, the ability to deliver disruptive UV radiation to virulent (viruses) microbes rather than potentially helpful and “good” bacteria allows for a much more effective and useful manner of protecting humans (and other mammals, at least) from viral infections, but also the ability to selectively do so without unnecessarily harming and killing certain microorganisms that are susceptible to kills from typical hand sanitizers and other metal (such as silver, for instance) based antimicrobials.

Thus, such a complete glove with UV-C LED integration therein provides the greatest sanitation/decontamination of surfaces picked up therewith, such as, without limitation, boxes, packages, mailings, papers, flatware and dishware, drinking vessels, remote controls, computers, keyboards, musical instruments, keys, arms, ammunition, furniture, grocery products, basically anything that may be held and/or transported while being manually held a/d/or carried with such a glove implement. As well, any surface that may be contacted with such an implement may be decontaminated/sanitized, as well, including, again, without limitation, table tops, floors, doors, doorknobs, windows, walls, steering wheels, dashboards, radar screens, computer screens, pilot controls, boat controls, furniture, staircases, railings, escalators, elevators, basically any surface that exists and may be contacted (including any carried articles as alluded to above) in such a manner. Such articles, products, surfaces, may further relate to anything repeatedly touched by multiple individuals, and may include, again, without limitation, anything related to supply chain and logistics concerns, as well. The list is thus endless and may help immeasurably in reducing the spread of microorganisms through passive as well as potentially active utilization thereof.

A standard inner cloth glove may be utilized, as well, with a vulcanization process to attach electronics (circuit board, and the like, flexible preferably) followed by the introduction of precut pieces of outer layer material with precut holes (approximately 1 mm in diameter, preferably) sized to be less than the UV-C LED diameter which can then be attached using a second vulcanizing process. Then the outer component edges can be stitched in place, allowing for multiple points of attachment of critical parts without limiting movement capability for the user/wearer. Alternatively, the outer layer may include openings through which the LEDs may be inserted to extend outwardly (in order to provide the necessary emissions for decontamination purposes). Such openings are thus filled by the LEDs in a fashion that prevents moisture/water passage, particularly in combination with the outer layer materials exhibiting such moisture-resistant properties. These different structural configurations provide shielding of the electronic components from moisture and the individual user/wearer from generated heat from the LEDs, as well as protection from sharp edges and electrical conduction. Such a glove can thus further protect a user/wearer from having to touch his or her face during use thereof as such a glove will not burn skin but still can not only allow for sanitation of any touched body areas, but also continuously decontaminates itself to prevent any introduction of microorganisms in such a manner to any other surface (including one's own face). The hands may easily infect surfaces therefore providing the opportunity for pathogen transmission to noninfected individuals. Removing the transmission vector is key, which is accomplished with this glove device. Existing air cleansing systems can provide air cleaning. Simple cloth masks reduce direct droplet transmission to just inches. The most dangerous vector which is the hand which infected surfaces and provides a transmission vector for the non-infected individual who touches an infected surface and then touches their face is removed using self-sanitizing gloves with UV-C LEDs as now disclosed.

As it concerns the preferred distance of UV-C LED exposure to contaminated surfaces, a 1 millimeter (mm) to 1 centimeter (cm), preferably from 1 to 3 millimeters, is workable, particularly to reduce the chances of harm to users, as well as reducing the amount of power required to produce maximum kill rates with lowered potential for user injury. Certainly, the closer the proximity to the target surface, the better for such a purpose (thus 1 mm is preferred for such a reason, limiting the potential for escape of UV-C emissions due to the glove being so close to and placed or even pressed downward thereto). Furthermore, with pressure applications, as noted above, the LEDs may activate (tactile pressure sensors, again) and remain on for a sufficient time to deliver such emissions for maximum kill rates. As such, a range of 3 seconds activation time to as much as 4 (or more) minutes, from 3 seconds to 2 minutes, and so on, may be permitted before the MCU (flexible circuit board “brains” of the glove device) automatically causes shut down, particularly if pressure does not continue. Such a range of activation times is necessary to ensure the MCU does not continue turn on/off continuously (such as if a user applies pressure, lifts it up, then again applies pressure to a surface in a repetitive, if not also haphazard sequence). The ability to remain on thus allows for the MCU to not experience too much in the way of activation/deactivation to prolong useful life thereof such a glove as well as reduce heat generated thereby unnecessarily, allowing for greater life as well as maximum comfort for the user.

The gloves may thus be provided within a complete kit for a full UV-C decontamination box concept, including such gloves, charging capability therefore such gloves, as well as a means (through the gloves or a sanitizing box) to sanitize a soft cloth mask, and UV-C eyewear protection as a precaution to prevent eye damage if the UV-C gloves were used improperly and/or haphazardly.

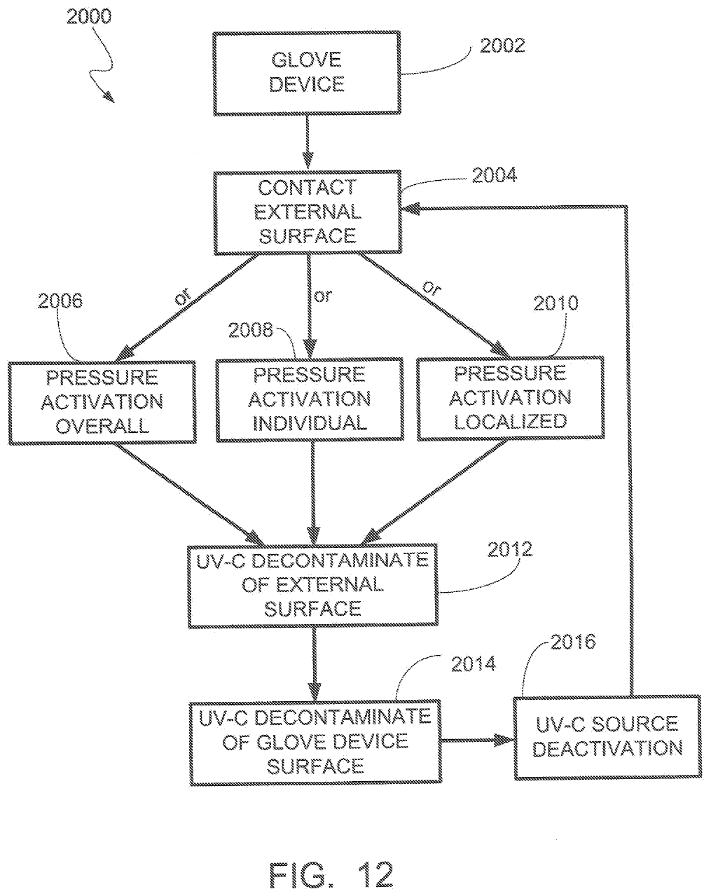

FIG. 12 shows a flow chart for the utilization of a glove device system 2000. A first step is the provision of a glove device 2002 (as noted in any of FIGS. 7-11, above) followed by contact with a subject external surface 2004 that activates the UV-C light source of the glove device 2006 (either in total across the entirety of the device 2008, or individually as pressure is sensed at each UV-C light source location 2010, or within a region associated with a group of UV-C light sources and a pressure sensor therein). Such activation thus allows for exposure and decontamination of the contacted external surface 2012 and simultaneously and subsequently the surface of the glove device 2014.

Thus, provided herein is a glove device to provide complete capabilities of decontamination of any type of surface with any type of suitable wearable or manipulatable glove. Such may be utilized for carrying boxes and materials, wiping hard surfaces (walls, tables, computer keyboards, etc., the list is endless), wiping food surfaces (including, for example, meat within slaughterhouses, and butcher shops, again the list is extremely long), floors, furniture, bathroom fixtures, kitchen sinks and counters, myriad things may be treated in such a manner, basically. Any surface that can be contacted by a person or object may also be incorporated and used with the base layered structured disclosed herein for decontamination capabilities. Such disclosed glove devices provide complete LED-based UV-C decontamination methods and procedures that undertake the maximum amount of decontamination capabilities with maximum safety and controlled power outputs for reliable microorganism kills, human safety, and comfort for users and bystanders.

It should be understood that various modifications within this disclosure's scope can be made by one of ordinary skill in the art without departing from the spirit thereof. Therefore, it is wished that this disclosure be defined by the scope of the appended claims as broadly as the prior art will permit and given the specification if need be.

1. A wearable glove device comprising a plurality of light emitting diodes embedded therein to provide external and surface exposure to UV-C radiation between 240 and 300 nm wavelengths, said glove further comprising an external surface water-proof, substantially nonporous, and alternatively isopropyl alcohol-resistant, material through which said plurality of light emitting diodes extend outwardly; and
wherein said external surface material exhibits a tensile strength of at least 5,000 psi.

2. The glove device of claim 1 wherein said glove comprises at least one control component selected from the group of at least one flexible circuit, at least one MCU, and a combination thereof, wherein said at least one control component is programmable for control of duration of UV-C emissions duration, control of UV-C light source power levels, and control of activation of UV-C light sources in relation to pressure application on a surface by a user or close proximity location to an external surface.

3. The glove device of claim 1 wherein said glove comprises an inner layer of moisture wicking and/or heat shielding material for comfort and/or protection of the wearer.

4. The glove device of claim 3 wherein said glove comprises a pressure sensor component underneath said external material and above said inner layer material.

5. The glove device of claim 1 wherein said plurality of UV-C light emitting diodes are positioned on the surface thereof said glove wherein the entirety of said glove device surface is exposed to UV-C light emissions upon activation thereof.

6. A method of eradicating microbes from an object surface, said method comprising the steps of:
i) providing a wearable glove device, wherein said glove device comprises: a) a plurality of light emitting diodes embedded therein to provide external and surface exposure to UV-C radiation between 240 and 300 nm wavelengths, b) an external surface material through which said plurality of light emitting diodes extend outwardly, wherein said material is waterproof, exhibits a tensile strength of at least 20,000 psi, and provides a smooth outer layer of said glove, c) at least one pressure sensor component indicating application of pressure of said glove on said object surface, and d) at least one control component selected from the group of at least one flexible circuit, at least one MCU, and a combination thereof, wherein said at least one control component is programmable for control of duration of UV-C emissions duration, control of UV-C light source power levels, and control of activation of UV-C light sources in relation to pressure application on a surface by a user;
ii) contacting said glove device with said object surface;
wherein the voltage for light emitting diode UV-C light generation is limited to a 100 mW maximum.

7. The method of claim 6 wherein said plurality of light emitting diodes are positioned on said glove device surface to provide UV-C emissions over the entirety of said glove device surface during activation thereof.

8. A wearable virulence eradication system for surface treatments and self-decontamination thereof, said system comprising a multi-layer implement comprising: i) a water-proof, substantially nonporous, high tensile strength, smooth material outer layer having a plurality of UV-C light emitting components extending therefrom, wherein said material outer layer reflects low wavelength emissions from said UV-C light emitting components, ii) a lower layer comprising a pressure sensor component, iii) a flexible circuit layer, and iv) an inner fabric layer;
wherein said system provides UV-C light emitting component activation upon contact with an external surface through said pressure sensor component and transfer of electrical impulses through said flexible circuit, and wherein said UV-C light emitting components provide exposure to any contacted external surface as well as the surface of said wearable implement.
